(19) United States
(12) Patent Application Publication
(10) Pub. No.: US 2021/0100506 A1
(43) Pub. Date: Apr. 8, 2021

(54) TOTAL PATIENT MANAGEMENT SYSTEM USING SMART DIAPER

(71) Applicant: MONIT CORP., Seoul (KR)

(72) Inventors: Jaeho BAEK, Seoul (KR); Jaehoon SONG, Seoul (KR); Jaehyun LEE, Gwangju-si (KR); Seongcheol LIM, Gwangmyeong-si (KR); Juho KIM, Yongin-si (KR); Jayoung MOON, Gwangmyeong-si (KR)

(73) Assignee: MONIT CORP., Seoul (KR)

(21) Appl. No.: 17/036,994

(22) Filed: Sep. 29, 2020

Related U.S. Application Data
(63) Continuation of application No. PCT/KR2019/013041, filed on Oct. 4, 2019.

(51) Int. Cl. A61B 5/00; A61B 5/11; A61F 13/42

(52) U.S. Cl. CPC A61B 5/6808; A61B 5/0004; A61B 5/1116; A61B 2562/164; A61F 13/42; A61B 2562/08; A61B 5/0015

(57) ABSTRACT

A total patient management system according to an example embodiment includes a smart diaper configured to mount with a temperature sensor, a humidity sensor, a gas sensor, a capacitance sensor, a gyro sensor, a magnetic field sensor, and an acceleration sensor; a transmission device configured to transmit a sensor signal generated in the smart diaper; and a central processing device configured to analyze change information of the smart diaper and a defecation pattern of a wearer using the sensor signal received from the transmission device and to monitor posture information and abnormality of a patient.

### CROSS-REFERENCE TO RELATED APPLICATIONS

The present application is a continuation of International Patent Application No. PCT/KR2019/013041, filed on Oct. 4, 2019. The disclosure of the above application is hereby incorporated by reference herein in its entirety.

### TECHNICAL FIELD

At least one example embodiment relates to a total patient management system using a smart diaper, and more particularly, to a system that may monitor and manage a behavioral status, a location, and a defecation cycle of a patient using the smart diaper.

### RELATED ART

As Korea enters an aging society, a number of persons having difficulty in dealing with defecation by themselves is increasing and a number of patient caregivers in hospitals is gradually decreasing. In the case of a patient having a difficulty in resolving bowel movements, the patient is resolving an urgent defecation situation using a diaper.

However, a diaper requires assistance of a guardian for treatment after defecation. If a defecation treatment is not performed in a timely manner due to irregular defecation habit of a wearer and indifference of a guardian, skin rash or soreness may occur.

To resolve this issue, Korean Patent Registration No. 10-1700784, registered on Jan. 23, 2017, titled “patient management system using diaper for adult” describes a system including a detection sensor unit configured to detect presence of defection in a diaper for adult, a transmitter configured to transmit an identification (ID) number of the diaper through a wireless communication network, a receiver configured to receive a signal, and a notifier configured to transmits a diaper change signal to a guardian, such that a wearer of the diaper may have the diaper changed timely.

Also, U.S. Patent Laid-Open Publication No. 2019/0091073, published on Mar. 28, 2019, titled “diaper care system and method for multiple users” describes a system for informing a patent that requires change of a diaper by managing a plurality of diaper managers through a single control system.

However, a system for monitoring and managing a diaper user simply notifies the user of a diaper change timing from the perspective of a manager that manages a patient and does not provide detailed information regarding a level of defecation at which the patient does not feel uncomfortable or a level of defecation at which the patient feels uncomfortable and needs change of a diaper.

For a patient with disability, changing a diaper may also act as a stress. Therefore, it is more important than anything else to provide an optimal environment with the least possible change cycle.

Accordingly, to outperform the above issue, there is a need for a system for predicting a health status and a behavioral pattern of a patient through a diaper as well as a diaper management system capable of monitoring a defecation amount.

### Object

At least one example embodiment provides a total patient management system for informing a diaper change cycle.

### Solution

A total patient management system according to an example embodiment includes a smart diaper configured to mount with a temperature sensor, a humidity sensor, a gas sensor, a capacitance sensor, a gyro sensor, a magnetic field sensor, and an acceleration sensor, a transmission device configured to transmit a sensor signal generated in the smart diaper, and a central processing device configured to analyze change information of the smart diaper and a defecation pattern of a wearer using the sensor signal received from the transmission device and to monitor posture information and abnormality of a patient.

Also, the smart diaper may comprise a defecation amount measurement sensing unit that includes at least one temperature sensor, at least one humidity sensor, at least one gas sensor, and at least one capacitance sensor, and a wearing-and-posture sensing unit that includes the at least one capacitance sensor, at least one gyro sensor, at least one magnetic field sensor, and at least one acceleration sensor.

Also, the sensor signal may include a diaper serial number.

Also, the central processing device may comprise a user matcher configured to match the diaper serial number and user information; a defecation determiner configured to determine a type of defecation and presence or absence of defecation based on at least one piece of information selected from among temperature information, humidity information, capacitance information, and gas information of the sensor signal, a defecation amount determiner configured to determine an amount of defecation based on capacitance information of the sensor signal or posture information, and a storage configured to store a defecation pattern, a defecation amount, posture information, and a diaper change cycle of a user in temporal order.

Also, the central processing device may further comprise a change determiner configured to determine the amount of defecation using the defecation amount determiner and to determine whether a diaper change is required based on diaper change cycle information.

Also, the central processing device may further comprise a monitoring unit configured to determine whether the patient is in an abnormal status based on a change in a matched defecation pattern and defecation amount of the user.

A total patient management method performed by a central processing device according to another aspect includes receiving a sensor signal generated in a smart diaper, matching a diaper serial number included in the sensor signal and a patient, determining presence or absence of defecation and a type of defecation from the sensor signal, determining a defecation amount from the sensor signal, and determining whether a diaper change is required for the patient based on a previous defecation pattern, defecation amount, posture information, and diaper change cycle of the patient.

Also, the total patient management method according to an example embodiment may further include determining whether the patient is in an abnormal status based on the previous defecation pattern, defecation amount, posture information, and diaper change cycle of the patient.

### Effect

A total patient management system according to an example embodiment may monitor a defecation status of a patient, a defecation pattern of the patient, and a behavior of the patient, and through this, may continuously manage a condition of the patient.

Also, a total patient management system according to an example embodiment may measure an amount of defecation and thereby prevent an unnecessary change of a diaper or an unnecessary use of manpower.

Also, a total patient management system according to an example embodiment provides a timely manageable system regardless of a reduction in the management personnel.

### BRIEF DESCRIPTION OF THE DRAWINGS

FIG. 1 is a diagram illustrating a configuration of a total patient management system according to an example embodiment.

FIG. 2 illustrates an example of a capacitance sensor according to an example embodiment.

FIG. 3 illustrates an example of a capacitance sensor according to another example embodiment.

FIG. 4 illustrates an example of a smart diaper according to an example embodiment.

FIG. 5 illustrates a situation in which a plurality of transmission devices is installed according to an example embodiment.

FIG. 6 is a block diagram illustrating a detailed configuration of a central processing device according to an example embodiment.

FIG. 7 illustrates an example of an output screen about a patient monitoring result according to an example embodiment.

FIG. 8 is a flowchart illustrating a total patient management method according to an example embodiment.

### DETAILED DESCRIPTION

The features and effects of example embodiments will be apparent through the following detailed description described with reference to the accompanying drawings. Accordingly, those skilled in the art may easily implement the technical spirit of the present disclosure.

Various alterations and modifications may be made to the example embodiments and thus, specific example embodiments are illustrated as examples and are described in the detailed description. The example embodiments are not construed as being limited to the disclosure and should be understood to include all changes, equivalents, and replacements within the spirit and technical scope of the disclosure.

Regarding reference numerals assigned to elements in the drawings, like reference numerals refer to like elements.

Terms, such as first, second, and the like, may be used herein to describe components. Each of these terminologies is not used to define an essence, order, or sequence of a corresponding component but used merely to distinguish the corresponding component from other component(s).

For example, a first component may be referred to as a second component, and similarly, the second component may also be referred to as the first component without departing from the scope of the disclosure. As used herein, the term “and/or” includes any and all combinations of one or more of the associated listed items.

Unless otherwise defined, all terms, including technical and scientific terms, used herein have the same meaning as commonly understood by one of ordinary skill in the art to which this disclosure pertains.

Terms, such as those defined in commonly used dictionaries, are to be interpreted as having a meaning that is consistent with their meaning in the context of the related art, and are not to be interpreted in an idealized or overly formal sense unless expressly so defined herein.

Also, the suffixes “˜module/block/unit,” etc., used for the following components are assigned or used for ease of preparing the specification only and are not construed to have distinguishing meanings or roles.

Hereinafter, some example embodiments will be described in detail with reference to the accompanying drawings to be easily implemented by those skilled in the art. Also, in the description of example embodiments, detailed description of well-known related structures or functions will be omitted when it is deemed that such description will cause ambiguous interpretation of the present disclosure.

Hereinafter, a total patient management system using a smart diaper according to the present disclosure is described in detail.

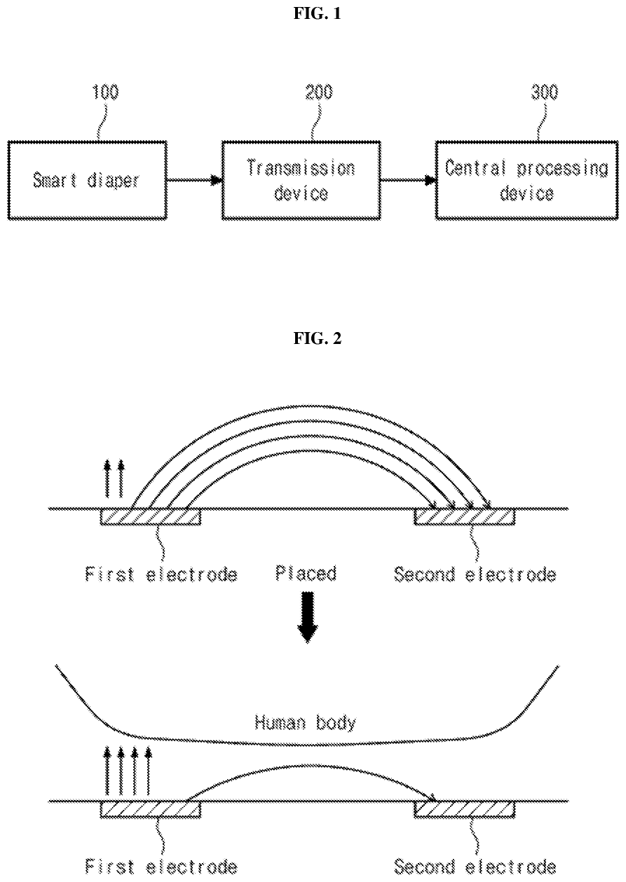

FIG. 1 is a diagram illustrating a configuration of a total patient management system according to an example embodiment.

Referring to FIG. 1, the total patient management system according to an example embodiment includes a smart diaper 100, a transmission device 200, and a central processing device 300.

The smart diaper 100 is mounted with a temperature sensor, a humidity sensor, a gas sensor, a capacitance sensor, a gyro sensor, a magnetic field sensor, and an acceleration sensor.

The temperature sensor detects a temperature of a detection area using an electronic device material of which an electrical characteristic varies according to temperature. The temperature sensor may be classified into a contact type and a noncontact type. The temperature sensor according to an example embodiment may be a noncontact temperature sensor, however, is not limited thereto.

The humidity sensor refers to a sensor that measures the moisture content in the air and may measure the moisture content based on a change in electrical resistance or capacitance that varies based on the moisture content absorbed through a porous ceramic or a polymer membrane. Desirably, the humidity sensor may be an integrated circuit (IC) humidity sensor in which a humidity sensor element and a signal processing electronic circuit are integrated.

The gas sensor may use various methods, such as, for example, a method of using a change in physical property of solid by absorption or reaction of gas, a method of using the heat of combustion, and a method of using an electrochemical reaction. Desirably, the gas sensor may be a semiconductor gas conductor that uses the method of using a change in physical property of solid by absorption or reaction of gas.

Gas generated from the defecation of a human body mainly includes ammonia, hydrogen sulfide, skatole, and indole in addition to nitrogen, carbon dioxide, and hydrogen, and the gas sensor detects volatile organic compounds (VOCs) generated in the urine and feces.

The capacitance sensor refers to a sensor that measures a change in capacitance occurring due to a potential difference between conductors (electrodes).

FIG. 2 illustrates an example of a capacitance sensor according to an example embodiment.

Referring to FIG. 2, in the case of determining presence or absence of boarding using the capacitance sensor in the smart diaper 100, the capacitance of the capacitance sensor varies. Further, even in the case in which a change in a dielectric material is detected in a path in which an electric field is formed between a first electrode and a second electrode, capacitance varies, and the capacitance sensor according to an example embodiment detects the change in the capacitance.

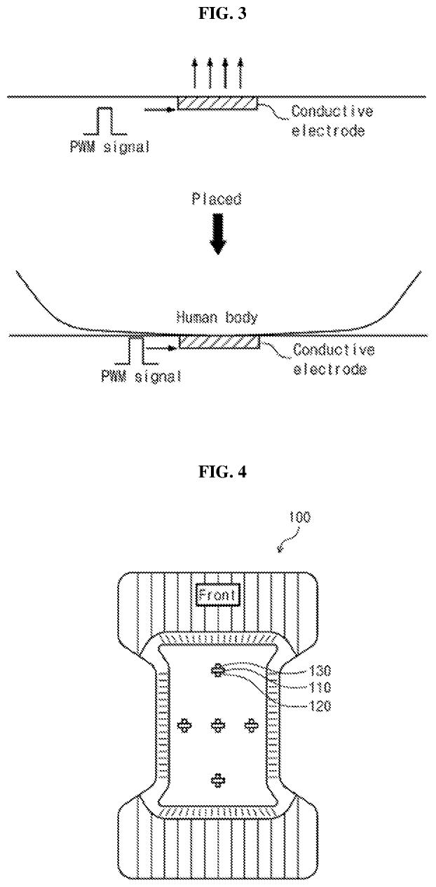

FIG. 3 illustrates an example of a capacitance sensor according to another example embodiment.

Referring to FIG. 3, the capacitance sensor according to another example embodiment detects a change in capacitance using a single conductive electrode. As shown in FIG. 3, if a pulse width modulation (PWM) signal is applied to a single conductive electrode, free electrons arranged on the surface of the electrode form an electric field on the surface of the electrode by the PWM signal.

Here, if the smart diaper (100) is placed on a user, a human body serves as a ground electrode and the free electrons arranged on the surface of the conductive electrode escape to the human body. Therefore, the electric field is not formed even by the PWM signal and whether the smart diaper is placed on the user may be detected.

The gyro sensor refers to an instrument that measures an angular velocity and measures an angle of rotation per hour in real time. The gyro sensor may accurately measure an amount of defecation having high fluidity by measuring a degree of inclination of the smart diaper.

The magnetic field sensor refers to a sensor configured to detect a magnetic field and sets reference locations of a value sensed at the gyro sensor and a value sensed at the acceleration sensor and corrects the sensed values. Further description is made below.

The acceleration sensor processes an output signal and measures the dynamic force, such as, for example, acceleration, vibration, impact, etc., of an object. The acceleration sensor mounted to the smart diaper is used to accurately measure the degree of inclination of the smart diaper 100 with the aforementioned gyro sensor.

FIG. 4 illustrates an example of a smart diaper according to an example embodiment.

Referring to FIG. 4, the temperature sensor, the humidity sensor, the gas sensor, the capacitance sensor, the gyro sensor, the magnetic field sensor, and the acceleration sensor mounted to the smart diaper 100 may be configured as a single sensor set. In particular, due to a characteristic of a device incapable of covering all of portions of a defecation area in the smart diaper with a single sensor set, a plurality of sensor sets may be classified into a male and a female and thereby installed at corresponding locations or areas on which the defecation is generally performed.

A single sensor set may be configured with a compact size and further, may be manufactured into a single chip. In this manner, a plurality of sensor sets may be installed on the smart diaper 100.

The smart diaper 100 may include a defecation amount measurement sensing unit 110 that includes at least one temperature sensor, at least one humidity sensor, at least one gas sensor, and at least one capacitance sensor and a wearing-and-posture sensing unit 120 that includes the at least one capacitance sensor, at least one gyro sensor, at least one magnetic field sensor, and at least one acceleration sensor.

The aforementioned temperature sensor, humidity sensor, gas sensor, and capacitance sensor may constitute the defecation amount measurement sensing unit 110 and provide first basic data used to predict presence or absence of defecation and an amount of defecation. The aforementioned capacitance sensor, gyro sensor, magnetic field sensor, and acceleration sensor may constitute the wearing-and-posture sensing unit 120 and provide second basic data used to predict whether the diaper (100) is placed on the patient and posture of the patient.

Also, at least one temperature sensor and humidity sensor may further include at least one sensor exposed on the outer surface of the diaper (100) to be capable of sensing the temperature and humidity of the outside.

The sensor capable of sensing the temperature and the humidity of the outside may be used to collect temperature and humidity information of an external environment in which the diaper (100) is placed on the patient and to predict presence or absence of defecation and an amount of defection in the diaper.

If the first basic data and the second basic data collected by various sensors installed on the smart diaper 100 are defined as sensor signals, each individual sensor signal includes a diaper serial number.

The diaper serial number includes information about, for example, a wearer (patient), a gender (male/female), and a bed location, and the like. Information of the patient is matched by inputting a label attached on the surface of the diaper or a wrapper of the diaper in a form of, for example, a barcode and a quick read (QR) code before placing the diaper on the patient or when taking out the diaper from the patient.

The smart diaper 100 may include a separate transmitter configured to transmit a sensed sensor signal to the transmission device 200 for each sensor set. The transmitter 130 may be connected to the transmission device 200 through a short range wireless communication network.

The transmitter 130 that constitutes the short range communication network with the transmission device 200 may include, for example, wireless fidelity (Wi-Fi), ZigBee, Bluetooth, Wi-Fi direct, near field communication (NFC), Bluetooth low energy (BLE), and controller area network (CAN) communication.

The transmission device 200 serves to receive the sensor signal transmitted from the smart diaper 100 and to transfer the sensor signal to the central processing device 300. The transmission device 200 may include a module capable of performing short range wireless communication and, at the same time, performing long range wired/wireless communication.

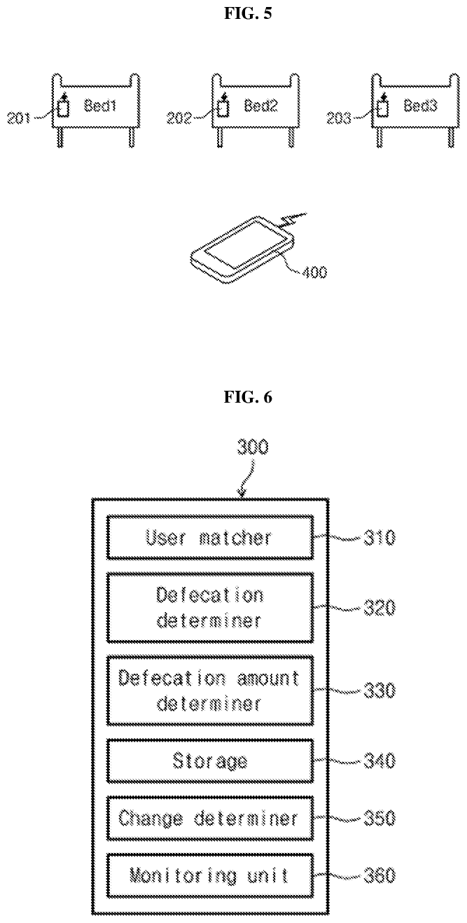

FIG. 5 illustrates a situation in which a plurality of transmission devices is installed according to an example embodiment.

A single transmission device 200 according to an example embodiment may be installed in a short range communication area to be capable of covering a space in which a plurality of patients is present and may transmit a sensor signal to the central processing device 300.

Also, referring to FIG. 5, according to another example embodiment, the transmission device 200 may be installed in each bed in which an individual patient is present and may receive a sensor signal transmitted from the smart diaper 100 and may transmit the sensor signal to a mobile terminal 400 of a manager.

For example, with the assumption that a plurality of transmission devices 201, 202, and 203 transmit a sensor signal while the manager is moving with the mobile terminal 400, the manager receives a sensor signal transmitted from the transmission device 202 most adjacent to the mobile terminal 400 and verifies information regarding whether a diaper change is required through the mobile terminal 400. Referring to FIG. 5, the mobile terminal 400 of the manager receives a sensor signal of a patient present in a bed (bed 2) in which the adjacent transmission device 202 is installed and outputs a message capable of verifying whether a diaper change is required and whether the patient is in an abnormal status according to the sensor signal.

Further, the transmission device 202 of FIG. 5 may simultaneously transmit a sensor signal to the mobile terminal 400 of the manager and the central processing device 300, and may transmit necessary information to the mobile terminal 400. To verify information, such as, checking whether the patient is in an abnormal status, an inference process needs to be performed based on prestored information of the patient. Therefore, if processing is actually impossible at the mobile terminal 400, a processing result of the central processing device 300 may be verified using the mobile terminal 400.

The transmission device 200 may constitute a short range communication network or a telecommunication network with the central processing device 300 and may transmit a sensor signal. The short range communication network may include, for example, Wi-Fi, ZigBee, Bluetooth, Wi-Fi direct, NFC, BLE, and CAN communication. The telecommunication network may include a communication network implemented based on a mobile communication standard, for example, 3rd generation partnership project (3GPP), 3GPP2, and World interoperability for Microwave Access (WiMAX) series, and may include a radio frequency (RF) network, a long term evolution (LTE) network, a WiMAX network, Internet, a local area network (LAN), a wireless LAN, a wide area network (WAN), a personal area network (PAN), a Bluetooth network, an NFC network, a satellite broadcasting network, an analog broadcasting network, a digital multimedia broadcasting (DMB) network, and the like, without being limited thereto.

Also, for information security, a private network may be used and a virtual private network (PVN) may also be used.

FIG. 6 is a block diagram illustrating a detailed configuration of a central processing device according to an example embodiment.

Referring to FIG. 6, the central processing device 300 includes a user matcher 310 configured to match the diaper serial number and user information, a defecation determiner 320 configured to determine a type of defecation and presence or absence of defecation based on at least one piece of information selected from among temperature information, humidity information, capacitance information, and gas information of the sensor signal, a defecation amount determiner 330 configured to determine an amount of defecation based on capacitance information of the sensor signal or posture information, and a storage 340 configured to store a defecation pattern, a defecation amount, posture information, and a diaper change cycle of the user in temporal order.

Also, the central processing device 300 includes a change determiner 350 configured to determine the amount of defecation using the defecation amount determiner 330 and to determine whether a diaper change is required based on diaper change cycle information stored in the storage 340, and includes a monitoring unit 360 configured to determine whether the patient is in an abnormal status based on a change in a matched defecation pattern and defecation amount of the user.

The user matcher 310 matches a diaper serial number included in an actual sensor signal transmitted to the central processing device 300 to a wearer (patient) using the diaper serial number input to the central processing device 300 at a time of taking out the diaper and information of a target user on which the diaper is placed.

In addition, once the patient is specified, the user matcher 310 may call information associated with the patient stored in the storage 340, which is described below.

The defecation determiner 320 determines presence or absence of defecation based on temperature information, humidity information, capacitance information, and gas information included in the sensor signal. In particular, temperature information and humidity information are used to determine presence or absence of defecation based on temperature information/humidity information acquired by periodically changing a threshold for determining the presence or the absence of defecation into consideration of temperature information and humidity information of the outside air that are periodically measured. The capacitance information is used to determine presence or absence of a foreign material, such as a defection material, by detecting a change in capacitance between at least two conductive electrodes in which a potential difference occurs.

A capacitor includes a dielectric between electrodes and a relative dielectric constant (εᵣ) varies due to a material change of the dielectric, a component change, and input of a foreign material. If the relative dielectric constant varies, the capacity of the capacitor, that is, capacitance varies.

A change in a dielectric constant of the dielectric between the electrodes of the capacitor caused by capacitance may be observed based on an electrical characteristic. Here, if a change in the observed capacitance in time series occurs, input of a foreign material related to defecation may be predicted.

The defecation amount determiner 330 determines an amount of defecation based on capacitance information of the sensor signal or posture information. Here, the capacitance information of the sensor signal may be used to infer a defecation amount according to pre-learned capacitance through a time series change in the capacitance. Also, the posture information refers to gyro sensor information, magnetic field sensor information, and acceleration sensor information included in the sensor signal.

To determine a defecation amount, a state of inclination of the smart diaper 100 is important. The defecation amount may be accurately determined through a sensor value of which sensor set is applied among the plurality of sensor sets installed in the smart diaper 100 based on the state of inclination.

The defecation amount determiner 330 predicts a posture of the user based on acceleration sensor information, magnetic field sensor information, and gyro sensor information. The defecation amount determiner 330 may predict a level of inclination of the smart diaper and the posture based on a roll value, a pitch value, and a yaw value measured by the gyro sensor. However, the gyro sensor triggers a phenomenon that an error occurs due to effect of the temperature and the error is accumulated and a final value is drifted.

Therefore, the gyro sensor compensates for the error using the temperature sensor. Also, from the standpoint of a long period of time in a stationary state, an angle of inclination calculated by the acceleration sensor exhibits a correct value and the gyro sensor exhibits a wrong value over time.

On the contrary, from the standpoint of a short period of time, the gyro sensor exhibits a correct value and the acceleration sensor may calculate a value different from an angle of inclination. Therefore, a defecation amount prediction module calculates a roll value, a pitch value, and a yaw value using an algorithm capable of compensating for an error using all of the acceleration sensor and the gyro sensor and infers the posture of the user.

Also, the magnetic field sensor calculates a roll value, a pitch value, and a yaw value operated by the gyro sensor, sets a center that is a standard of movement using the acceleration sensor, and enables a centric axis correction for each value. Accordingly, the posture of the user is inferred using the gyro sensor, the magnetic field sensor, and the acceleration sensor.

In the case of a lying patient, the defecation amount determiner 330 predicts an amount of defecation by synthesizing information, such as, for example, temperature, humidity, and capacitance at a sensor set installed at the respective individual locations as shown in FIG. 4.

The storage 340 stores information of a user specified by the user matcher 310 and information related to the defecation of the user. As the information related to the defecation, the storage 340 stores presence or absence of defecation, a type of defecation, and a time of defecation determined by the defecation determiner 320 and stores capacitance information, posture information (acceleration sensor information and gyro sensor information), and an amount of defecation in a time serial flow determined by the defecation amount determiner 330.

The storage 140 may include, for example, at least one of a main memory device and an auxiliary memory device. The main memory device may be implemented using a semiconductor storage medium, such as read only memory (ROM) and/or random access memory (RAM). In general, ROM may include typical ROM, erasable programmable read only memory (EPROM), electrically EPROM (EEPROM), and/or mask ROM. RAM may include, for example, dynamic random access memory (DRAM) and/or static RAM (SRAM). The auxiliary memory device may include at least one storage media capable of permanently or semi-permanently storing data, such as, for example, a flash memory device, a secure digital (SD) card, a solid state drive (SSD), a hard disc drive (HDD), a magnetic drum, optical media such as a compact disc (CD), a DVD, or a laser disc, a magnetic tape, a magneto-optical disc, and/or a floppy disc.

The change determiner 350 uses an amount of defecation determined by the defecation amount determiner 330 and information of a specific patient using the smart diaper (100) prestored in the storage (340). Here, the change determiner 350 determines whether a diaper change is required by considering a previous smart diaper change cycle of the patient and amount of defecation at a time of changing the smart diaper and transfers information to the manager.

The manager determines whether to change or maintain the diaper based on information transferred from the change determiner 350 of the central processing device 300 without the intention of the patient as to the change.

The monitoring unit 360 determines whether the patient is in an abnormal status based on a change in the matched defecation pattern and defecation amount of the user.

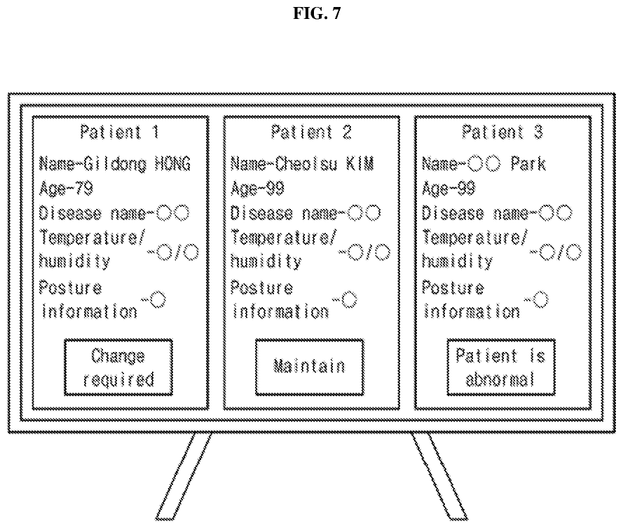

FIG. 7 illustrates an example of an output screen about a patient monitoring result according to an example embodiment.

The monitoring unit 360 determines whether the patient is in an abnormal status by matching information related to the defecation to the existing contents stored in the storage 340. Here, the monitoring unit 360 determines whether the patient is in an abnormal status by collectively considering, for example, a disease name, a behavioral status, and a posture change cycle of the patient.

For example, if an amount of defecation significantly increases or decreases compared to the past, the monitoring unit 360 determines that abnormality has occurred in a status of the patient and transmits a message to the manager. Also, a sudden decrease in the behavioral status or posture change of the patient may represent the occurrence in energy of the patient. Also, an irregular change in the number of defecations may represent the abnormality and thus, the monitoring unit 360 may determine that the patient is in an abnormal status.

Referring to FIG. 7, the manager may perform a total control through an output device capable of verifying a status of each patient wearing the smart diaper at a glance. Contents related to personal information, such as a name, an age, and a disease name of a corresponding patient, may be displayed on the output device, for example, a monitor. Here, temperature information/humidity information and posture information sensed at the smart diaper 100 in a current state may also be displayed on the monitor. The defecation determiner 320 and the defecation amount determiner 330 of the central processing device 300 determine presence or absence of defecation and a defecation amount using a sensor signal and output an alert message “Change required” as shown in Patient 1 of FIG. 7 if the change determiner 350 determines that a diaper change is required.

Also, referring to Patient 3, if there is a need to verify a status change and abnormality of the patient, the monitoring unit 360 may determine that a corresponding patient is in an abnormal status and output an alert message “patient is abnormal.”

A total patient management system according to an example embodiment may be configured to acquire presence or absence of defecation, an amount of defecation, and posture information of a patient using various types of sensors installed in the smart diaper 100, and to predict whether a diaper change is required and whether the patient is in an abnormal status based on the acquired information and to immediately inform the manager of whether diagnosis is necessary using the central processing unit 300.

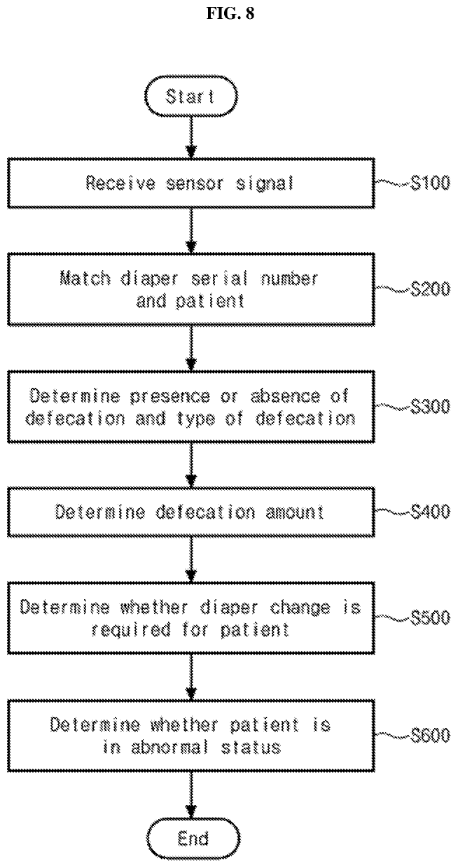

The total patient management system according to an example embodiment is described above. Hereinafter, a total patient management method according to another aspect is described with reference to FIG. 8.

Description related to a configuration similar to the aforementioned example embodiment is omitted.

FIG. 8 is a flowchart illustrating a total patient management method according to an example embodiment.

Referring to FIG. 8, the total patient management method according to an example embodiment may be performed by a central processing device and may include operation S100 of receiving a sensor signal generated in a smart diaper, operation S200 of matching a diaper serial number included in the sensor signal and a patient, operation S300 of determining presence or absence of defecation and a type of defecation from the sensor signal, operation S400 of determining a defecation amount from the sensor signal, operation S500 of determining whether a diaper change is required for the patient based on a previous defecation pattern, defecation amount, posture information, and diaper change cycle of the patient, and operation S600 of determining whether the patient is in an abnormal status based on the previous defecation pattern, defecation amount, posture information, and diaper change cycle of the patient.

Operation S100 of receiving the sensor signal refers to an operation of receiving time series data generated from a temperature sensor/humidity sensor, a capacitance sensor, an acceleration sensor, and a gyro sensor mounted to the smart diaper. As described above with reference to the example embodiment, a sensor signal may be transmitted from a transmission device through a short range, long range wired/wireless communication network. A serial number of the smart diaper may be assigned to the sensor signal.

In operation S200 of matching the diaper serial number included in the sensor signal and the patient, the diaper serial number is used to specify a wearing patient through information input at a time of taking out the diaper from the patient that requires a diaper changer and the patient is matched through the diaper serial number included in the sensor signal. If the patient is matched, stored information, such as, personal information and a previous defecation pattern of the patient, may be called.

In operation S300 of determining presence or absence of defecation and a type of defecation from the sensor signal, presence or absence of defecation and a type of defecation are determined based on time series temperature information, humidity information, capacitance information, and gas information included in the sensor signal. Prior thereto, a time at which the smart diaper is placed on the patient may be specified based on capacitance information. Also, temperature and humidity of the outside air at a time at which the smart diaper is placed on the patient may be measured and a threshold temperature/humidity may be set to determine the defecation.

In operation S400 of determining a defecation amount from the sensor signal, if the defecation is determined to be present, an amount of defecation is predicted through the capacitance sensor. Here, information about each of a plurality of sensor sets installed in the smart diaper is received and an amount of defecation is predicted based on inclination information of the smart diaper using the acceleration sensor and the gyro sensor.

In operation S500 of determining whether a diaper change is required for the patient based on a previous defecation pattern, defecation amount, posture information, and diaper change cycle of the patient, whether a diaper change is required is determined based on an actual defecation amount of the patient and diaper change history according thereto. Here, if the diaper change is determined to be required, a message “Change required” as shown in Patient 1 of FIG. 7 is output.

In operation S600 of determining whether the patient is in an abnormal status based on the previous defecation pattern, defecation amount, posture information, and diaper change cycle of the patient, if a difference between current information acquired from the time series sensor signal and previous information is greater or if a pattern itself varies, the patient is determined to be in an abnormal status and an alert message “patient is abnormal” is output as shown in Patient 3 of FIG. 7.

What is claimed is:

1. A total patient management system comprising:
a smart diaper configured to mount with a temperature sensor, a humidity sensor, a gas sensor, a capacitance sensor, a gyro sensor, a magnetic field sensor, and an acceleration sensor;
a transmission device configured to transmit a sensor signal generated in the smart diaper; and
a central processing device configured to analyze change information of the smart diaper and a defecation pattern of a wearer using the sensor signal received from the transmission device and to monitor posture information and abnormality of a patient.

2. The total patient management system of claim 1, wherein the smart diaper comprises:
a defecation amount measurement sensing unit that includes at least one temperature sensor, at least one humidity sensor, at least one gas sensor, and at least one capacitance sensor; and
a wearing-and-posture sensing unit that includes the at least one capacitance sensor, at least one gyro sensor, at least one magnetic field sensor, and at least one acceleration sensor.

3. The total patient management system of claim 1, wherein the sensor signal includes a diaper serial number.

4. The total patient management system of claim 3, wherein the central processing device comprises:
a user matcher configured to match the diaper serial number and user information;
a defecation determiner configured to determine a type of defecation and presence or absence of defecation based on at least one piece of information selected from a group including temperature information, humidity information, capacitance information, and gas information of the sensor signal;
a defecation amount determiner configured to determine an amount of defecation based on capacitance information of the sensor signal or posture information; and
a storage configured to store a defecation pattern, a defecation amount, posture information, and a diaper change cycle of a user in temporal order.

5. The total patient management system of claim 4, wherein the central processing device further comprises:
a change determiner configured to determine the amount of defecation using the defecation amount determiner and to determine whether a diaper change is required based on diaper change cycle information.

6. The total patient management system of claim 4, wherein the central processing device further comprises:
a monitoring unit configured to determine whether the patient is in an abnormal status based on a change in a matched defecation pattern and defecation amount of the user.

7. A total patient management method performed by a central processing device, the method comprising:
(a) receiving a sensor signal generated in a smart diaper;
(b) matching a diaper serial number included in the sensor signal and a patient;
(c) determining presence or absence of defecation and a type of defecation from the sensor signal;
(d) determining a defecation amount from the sensor signal; and
(e) determining whether a diaper change is required for the patient based on a previous defecation pattern, defecation amount, posture information, and diaper change cycle of the patient.

8. The method of claim 7, further comprising:
(f) determining whether the patient is in an abnormal status based on the previous defecation pattern, defecation amount, posture information, and diaper change cycle of the patient.

9. A recording medium storing a computer program to perform the method according to claim 7.

10. A recording medium storing a computer program to perform the method according to claim 8.
